(19) United States
(12) Patent Application Publication
(10) Pub. No.: US 2015/0173932 A1
(43) Pub. Date: Jun. 25, 2015

(54) PELVIC TRAUMA DEVICE

(71) Applicant: THE UNITED STATES OF AMERICA AS REPRESENTED BY THE SECRETARY OF THE ARMY, Fort Detrick, MD (US)

(72) Inventor: Howard Harcke, JR., Wilmington, DE (US)

(21) Appl. No.: 14/413,347

(22) PCT Filed: Aug. 4, 2013
(86) PCT No.: PCT/US2013/053531

§ 371 Date: Jan. 7, 2015

Related U.S. Application Data
(60) Provisional application No. 61/742,568, filed on Aug. 13, 2012.

(51) Int. Cl. A61F 5/058

(52) U.S. Cl. CPC A61F 5/058; A61F 2250/001

(57) ABSTRACT

A pelvic trauma device for emergency treatment of pelvic fracture includes two parallel elongated straps or belts that are connected, but are adjusted independently of each other. The two parallel belts include a lower belt and an upper belt. The two parallel belts are independently tensioned to provide: 1) pelvic fracture stabilization (lower belt); and 2) compression over the sacrum and anterior abdomen (upper belt).

### CROSS-REFERENCE TO RELATED APPLICATIONS

The present application claims the benefit of priority of U.S. provisional patent application Ser. No. 61/742,568 filed on Aug. 13, 2012, which is hereby expressly incorporated by reference.

### STATEMENT OF GOVERNMENT INTEREST

The invention described herein may be manufactured, used and licensed by or for the United States Government.

### BACKGROUND OF THE INVENTION

The invention relates to emergency treatment of a fractured pelvis.

A fractured pelvis may result from, for example, blunt force and/or blast injury. Pelvic fractures are a common injury pattern in casualties caused by Improvised Explosive Devices (IEDs). Internal bleeding caused by a fractured pelvis can easily result in death. The reduction of a fractured pelvis soon after injury substantially decreases mortality. Devices are known for reducing and stabilizing a fractured pelvis in emergency situations. After the pelvis is reduced and stabilized, the patient can be transported to a hospital or other treatment facility. Stabilization of the pelvis within the first hour after a fracture occurs is very important and may determine whether the patient lives or dies.

Known pelvic stabilization devices are disclosed in U.S. Pat. Nos. 6,554,784 and 7,008,389 issued to Krieg et al. and in U.S. Pat. No. 8,192,383 issued to Polliack et al. U.S. Pat. Nos. 6,554,784; 7,008,389; and 8,192,383 are expressly incorporated by reference herein. The known devices may be used to encircle the hips of an injured person and provide hoop tension to urge the parts of a person's fractured pelvic ring toward a normal relationship. Vascular damage from hemorrhage is associated with pelvic fracture and can be the fatal event. The known devices are designed to stabilize a fractured pelvis but do not specifically address pelvic hemorrhage associated with fractures, except for some possible tamponade with fixation.

Even when the known devices are properly used, fatal internal bleeding may occur. Postmortem angiograms have revealed pooling of blood at the posterior sacrum. In some cases, blood is pooled in the potential space between muscle and subcutaneous tissue and migrates cephalad. Bleeding may occur as a result of laceration of branches of the internal iliac arteries which perforate the sacral ala and are torn by fractures of the sacrum. Existing devices are placed over the femoral trochanters and are below the level of this blood accumulation.

A need exists for an emergency pelvic trauma device that more effectively treats pelvic hemorrhaging.

### SUMMARY OF THE INVENTION

One aspect of the invention is a pelvic trauma device having first and second belts. The first belt includes a first elongate strap having a central longitudinal axis and opposing end portions, and a padded enlarged portion having a greater width transverse to the central longitudinal axis than the first elongate strap. A first fastener is disposed at at least one of the opposing end portions of the first belt for maintaining a first tension in the first belt.

A second belt includes a second elongate strap having a central longitudinal axis, opposing end portions, and a padded enlarged portion having a greater width transverse to the central longitudinal axis than the second elongate strap. The central longitudinal axes of the first and second elongate straps are parallel. A second fastener is disposed at at least one of the opposing end portions of the second belt for maintaining a second tension in the second belt. The second tension is independent of the first tension.

The first and second belts are joined only along a portion of their respective padded enlarged portions. The joined portion is no more than one-half the overall length of the longer one of the padded enlarged portions of the first and second belts.

A posterior pad is disposed on only a part of the padded enlarged portion of the first belt. The posterior pad includes padding in addition to padding of the padded enlarged portion of the first belt. The posterior pad may have the shape of a trapezoid. The posterior pad may be centered on the longitudinal midpoint of the padded enlarged portion of the first belt.

An anterior pad may be disposed on the first elongate strap. The anterior pad may be longitudinally movable along the first elongate strap. The anterior pad may include a slot in which the first elongate strap slides.

Another aspect of the invention is a method that includes providing the novel pelvic trauma device, placing the lower belt around the greater trochanteric region of the pelvis of a person, and tightening the lower belt. The method includes placing the upper belt around the person at a level of the sacrum and anterior iliac crests and tightening the upper belt independently of tightening the lower belt.

The method may include positioning the posterior pad over the person's posterior sacrum and positioning the anterior pad about 180 degrees from the posterior pad in a midline of an abdomen of the person.

The invention will be better understood, and further objects, features and advantages of the invention will become more apparent from the following description, taken in conjunction with the accompanying drawings.

### BRIEF DESCRIPTION OF THE DRAWINGS

In the drawings, which are not necessarily to scale, like or corresponding parts are denoted by like or corresponding reference numerals.

FIG. 1A is an elevation view of the inner side of one embodiment of a pelvic trauma device.

FIG. 1B is an elevation view of the outer side of the device of FIG. 1A.

FIGS. 2A and 2B are front and rear views, respectively, of the pelvic area of a human skeleton.

FIG. 3 schematically shows the position of the pelvic trauma device on the pelvic area in FIG. 2A.

FIG. 4 is a side view of an anterior compression pad.

FIG. 5 is a front view of the abdominal area of a human.

### DETAILED DESCRIPTION

A novel pelvic trauma device is used for emergency treatment of pelvic trauma when fracture is suspected. The novel device addresses pelvic hemorrhage, which is not specifically addressed by prior devices. The novel pelvic trauma device includes two parallel elongated straps or belts that are joined to each other, but are adjusted independently of each other. The two parallel belts include a lower belt and an upper belt. The two parallel belts are independently tensioned to provide: 1) pelvic fracture stabilization (lower belt); and 2) compression over the sacrum and anterior abdomen (upper belt).

The lower or stabilization belt is placed at the level of the femoral greater trocanters. The upper or compression belt is placed at the sacral level. The upper belt includes a posterior compression pad that presses on the soft tissue over the posterior sacrum. The posterior compression pad is preferably trapezoidal in shape to fill the natural depression in the low back between the iliac wings. The upper belt may also include an anterior compression pad that compresses the bifurcation of the abdominal aorta and iliac arteries, thereby reducing blood flow to the more distal damaged arterial branches. Because the upper and lower belts are independently adjustable, the tension of the lower belt can be adjusted for optimum stabilization and the tension of the upper belt can be adjusted for optimum compression on the posterior sacrum.

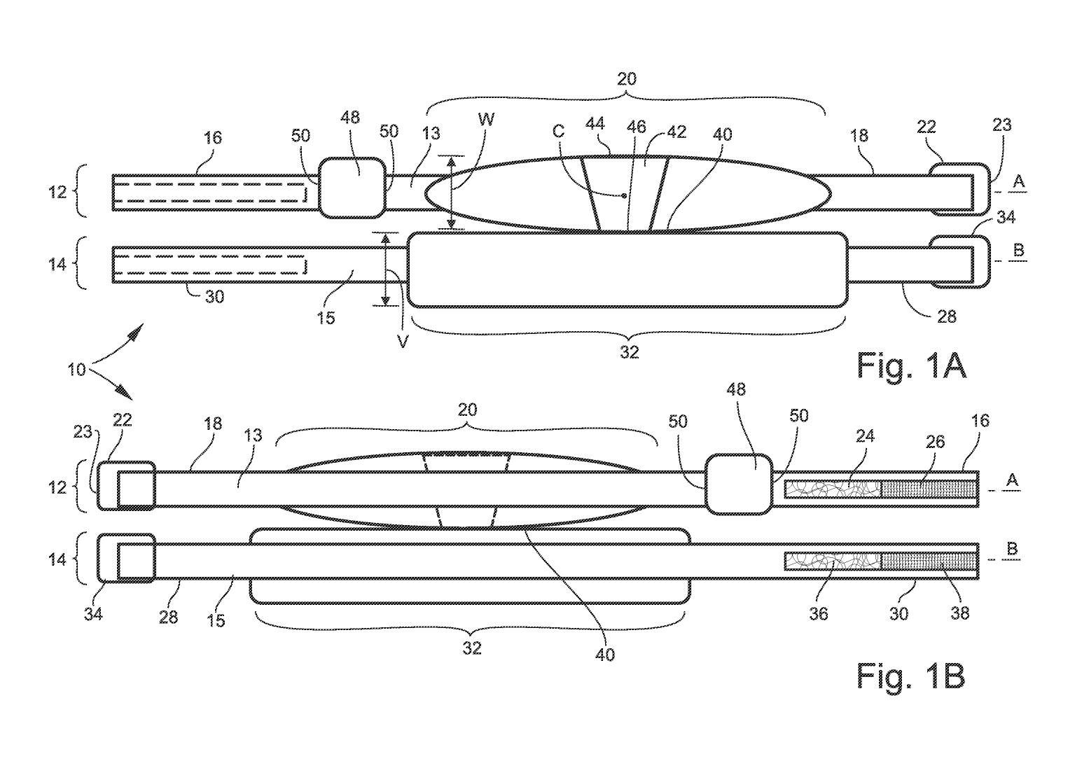

FIG. 1A is an elevation view of the inner side of one embodiment of a pelvic trauma device 10. The inner side is the side that contacts the patient. FIG. 1B is an elevation view of the outer side of device 10. Device 10 includes upper and lower belts 12, 14. “Upper” and “lower” refer to the placement of the belts on a human. The “upper” belt is nearer the head of a human than the “lower” belt, as is discussed in more detail below. Upper belt 12 is a primarily a compression belt and lower belt 14 is primarily a stabilization belt.

Upper belt 12 includes an elongate strap 13 having a central longitudinal axis A and opposing end portions 16, 18. An enlarged portion 20 is disposed on strap 13 and has a greater width W transverse to central longitudinal axis A than strap 13. A fastener is disposed at at least one of the opposing end portions 16, 18 of strap 13. The fastener maintains tension in upper belt 12. In the embodiment of FIGS. 1A-B, the fastener on upper belt 12 includes a buckle 22 fixed to end portion 18. The fastener also includes, on the outer side of end portion 16, one half 24 of a hook and loop fastener adjacent the other half 26 of a hook and loop fastener.

Lower belt 14 includes an elongate strap 15 having a central longitudinal axis B and opposing end portions 28, 30. An enlarged portion 32 is disposed on strap 15 and has a greater width V transverse to central longitudinal axis B than strap 15. Axes A and B are parallel. A fastener is disposed at at least one of the opposing end portions 28, 30 of strap 15. The fastener maintains tension in lower belt 14. In the embodiment of FIGS. 1A-B, the fastener on lower belt 14 includes a buckle 34 fixed to end portion 28. The fastener also includes, on the outer side of end portion 30, one half 36 of a hook and loop fastener adjacent the other half 38 of a hook and loop fastener.

Upper belt 12 and lower belt 14 are joined to each other along a portion 40 of their respective enlarged portions 20, 32. Enlarged portions 20, 32 may be joined at their edges by, for example, sewing. Each enlarged portion 20, 32 has an overall length along respective longitudinal axes, A, B. The overall lengths of enlarged portions 20, 32 are preferably about the same, although one enlarged portion may be longer than the other enlarged portion. The length of the joined portion 40 is no more than one-half the overall length of the longer of the enlarged portions 20, 32. Preferably, the length of joined portion 40 is no more than one-third of the overall length of the longer of the enlarged portions 20, 32. Joined portion 40 prevents belts 12, 14 from separating when device 10 is applied to a patient. However, the tension in lower belt 14 is independent of the tension in upper belt 12. The tension in lower belt 14 can be adjusted separate from the tension in upper belt 12.

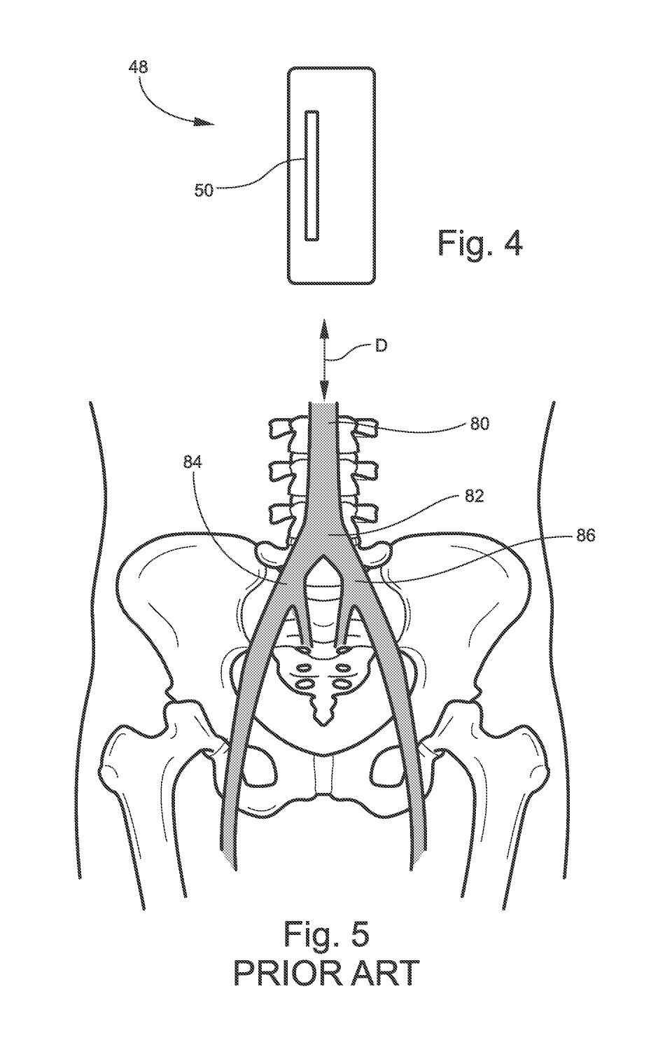

Upper belt 12 includes a posterior compression pad 42 disposed on enlarged portion 20. Pad 42 is preferably trapezoidal in shape, with the longer side 44 of the two parallel sides 44, 46 of the trapezoid disposed above the shorter side 46, as shown in FIG. 1A. Preferably, pad 42 is centered on a longitudinal midpoint C of enlarged portion 20. An anterior compression pad 48 may be disposed on elongated strap 13 of upper belt 12. Pad 48 is preferably longitudinally movable along elongate strap 13. Pad 48 may include a slot 50 (FIG. 4) formed in its thickness. Elongate strap 13 may slide through slot 50 to longitudinally position pad 48 on strap 13. Pad 48 may be generally rectangular-shaped.

Elongate straps 13, 15 may be made of a variety of materials, such as, for example, nylon webbing. Enlarged portions 20, 32 may be made of a variety of materials, such as, for example, felt padding. Pads 42, 48 may be made of a variety of materials, such as, for example, wool or synthetic fiber batting.

Device 10 may be made in different sizes to accommodate different size human beings. In one embodiment of device 10, elongate straps 13, 15 are about two inches wide and about 54 inches long; enlarged portion 20 of upper belt 12 is an oval shaped felt pad with a major (longitudinal) dimension of about twenty-four inches, a minor (transverse) dimension of about six inches, and a thickness of about 0.625 inches; enlarged portion 32 of lower belt 14 is a rectangular shaped felt pad about six inches by 24 inches and about 0.625 inches thick; posterior pad 42 is a trapezoid with parallel sides 44, 46 having lengths of about five and two inches, respectively, sides 44, 46 are about six inches apart and pad 42 is about 0.75 inches thick; and anterior pad 48 is a rectangular shape that is about 4.5 inches by six inches and about two inches thick. The joint at portion 40 may be, for example, about six inches long.

Upper belt 12 is tightened by passing end portion 16 through buckle 22 and then pulling end portion 16 in a reverse direction around side 23 of buckle 22 so that the halves 24, 26 of the hook and loop fastener on the outer side of strap 13 can be pressed together. Lower belt 14 is tightened in a similar manner. The fasteners for belts 12, 14 shown in the embodiment of FIGS. 1A-B are exemplary only. Other types of fasteners that can maintain tension in belts 12, 14 may be used. For example, the buckle described in U.S. Pat. No. 7,008,389 may be used in lieu of buckles 22, 34 and hook and loop fasteners 24, 26 and 36, 38. Or, the tensioning system described in U.S. Pat. No. 8,192,383 could be used with device 10.

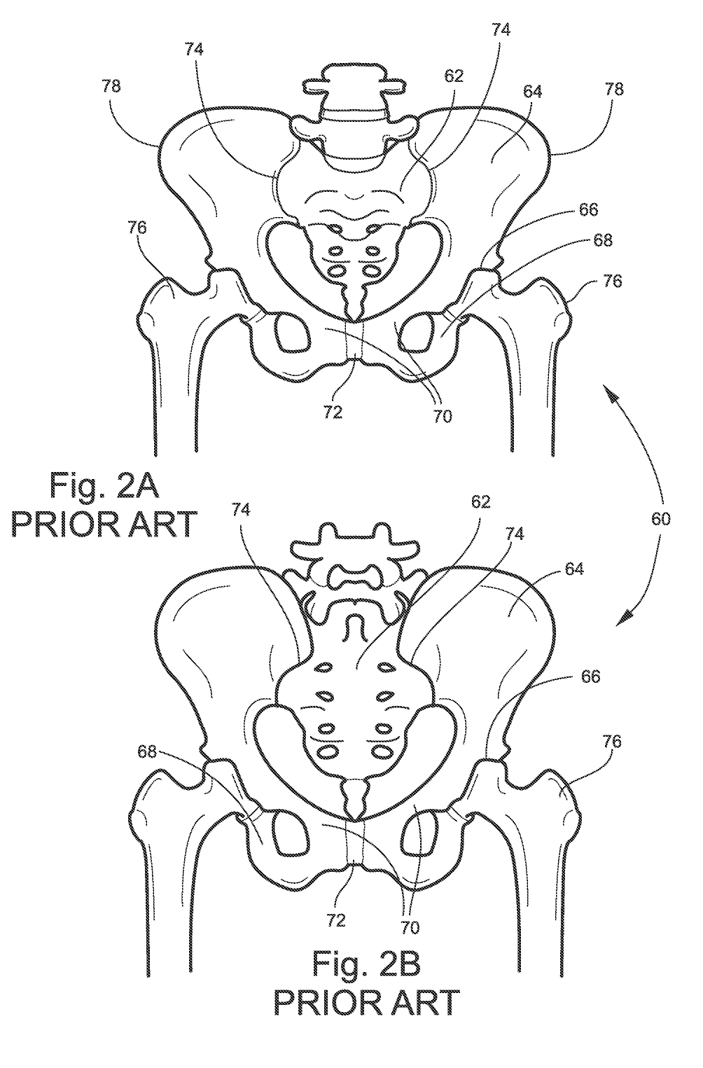

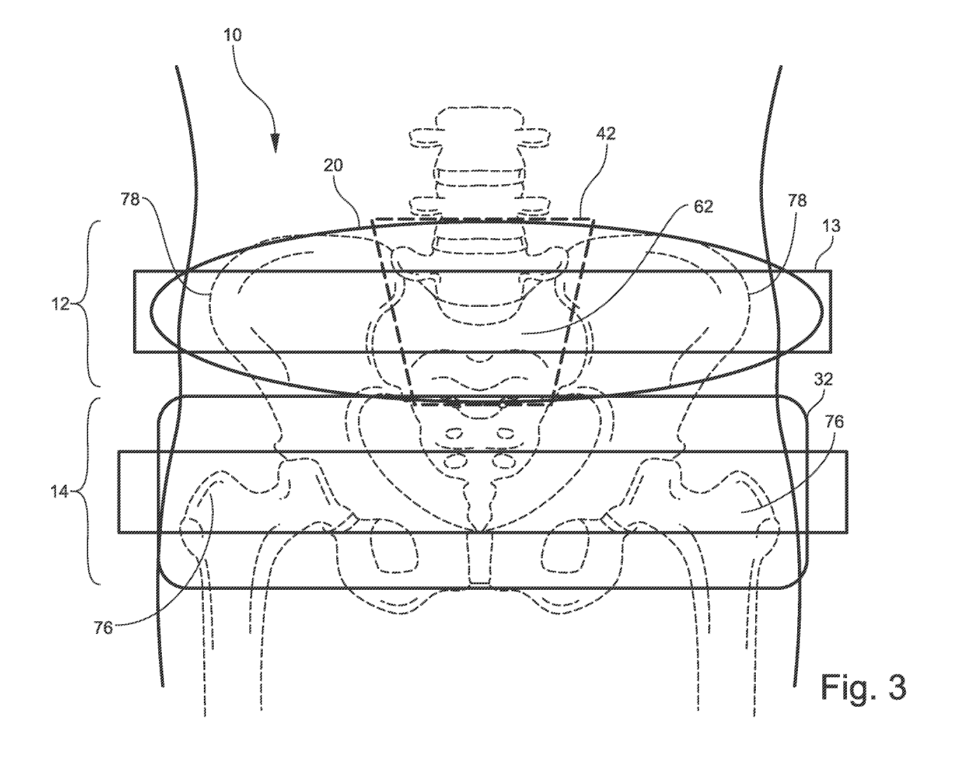

FIGS. 2A and 2B are front and rear views, respectively, of the pelvic area of a human skeleton 60. The pelvic ring is formed by the sacrum 62, ilium 64, acetabulum 66, ischium 68, pubic rami 70, and symphysis pubis 72. Posteriorly (FIG. 2B), the pelvic ring contains the sacroiliac joints 74, which connect the sacrum 62 with the left and right ilium 64. Reference numeral 76 is the greater trochanter of the femur.

Device 10 is placed on a human suspected of pelvic trauma. FIG. 3 is a schematic representation of device 10 positioned on the pelvic area shown in FIG. 2A. Lower or stabilization belt 14 encircles the human at the level of the femoral greater trocanters 76. Enlarged portion 32 of lower belt 14 is arranged symmetrically on the posterior of the human. Upper or compression belt 12 encircles the human at the sacral level such that posterior pad 42 fills the natural depression in the low back between the iliac wings. Upper belt 12 should be at the level of the anterior iliac crests 78.

Enlarged portion 20 of upper belt 12 is arranged symmetrically on the posterior of the human. Anterior pad 48 (not shown in FIG. 3) is adjusted on strap 13 to be about 180 degrees from posterior pad 42. Referring to FIG. 5, anterior pad 48 is positioned on the abdominal midline D so that pad 48 is centered over the aortic bifurcation area 82 where the abdominal aorta 80 bifurcates to form the right and left common iliac arteries 84, 86. The belts 12, 14 are then independently tightened, as discussed above.

It will be understood that many additional changes in the details, materials, steps and arrangement of parts, which have been herein described and illustrated in order to explain the nature of the invention, may be made by those skilled in the art within the principle and scope of the invention, as expressed in the appended claims.

1. A pelvic trauma device, comprising:
a first belt and a second belt;
the first belt including a first elongate strap having a central longitudinal axis and opposing end portions, and a padded enlarged portion having a greater width transverse to the central longitudinal axis than the first elongate strap;
a first fastener that is disposed at at least one of the opposing end portions of the first belt for maintaining a first tension in the first belt;
a posterior pad disposed on only a part of the padded enlarged portion of the first belt, the posterior pad including padding in addition to padding of the padded enlarged portion of the first belt, the posterior pad being centered on a longitudinal midpoint of the padded enlarged portion of the first belt;
a second belt including a second elongate strap having a central longitudinal axis, opposing end portions, and a padded enlarged portion having a greater width transverse to the central longitudinal axis than the second elongate strap , the central longitudinal axes of the first and second elongate straps being parallel; and
a second fastener that is disposed at at least one of the opposing end portions of the second belt for maintaining a second tension in the second belt, the second tension being independent of the first tension;
wherein the first and second belts are joined only along a portion of their respective padded enlarged portions, the joined portion being no more than one-half an overall length of a longer one of the padded enlarged portions of the first and second belts.

2. The device of claim 1, wherein the joined portion is no more than one-third the overall length of the longer one of the padded enlarged portions of the first and second belts.

3. (canceled)

4. The device of claim 1, wherein the posterior pad has a shape of a trapezoid.

5. The device of claim 1, further comprising an anterior pad disposed on the first elongate strap about 180 degrees from the posterior pad.

6. The device of claim 5, wherein the anterior pad is longitudinally movable along the first elongate strap.

7. The device of claim 6, wherein the anterior pad includes a slot and the first elongate strap slides through the slot.

8. The device of claim 7, wherein the anterior pad is generally rectangular-shaped.

9. The device of claim 1, wherein an entirety of the padded enlarged portion of the first belt is made of a padded material.

10. The device of claim 9, wherein an entirety of the padded enlarged portion of the second belt is made of a padded material.

11. The device of claim 1, wherein the first fastener includes a first buckle fixed to one of the opposing end portions of the first elongate strap and a first hook and loop fastener fixed to an outer side of another of the opposing end portions of the first elongate strap.

12. The device of claim 11, wherein the first hook and loop fastener includes two halves, the two halves being disposed longitudinally apart on the first elongate strap such that when the outer side of the other of the end portions is folded over, the two halves are fixed to each other.

13. The device of claim 11, wherein the second fastener includes a second buckle fixed to one of the opposing end portions of the second elongate strap and a second hook and loop fastener fixed to an outer side of another of the opposing end portions of the second elongate strap.

14. The device of claim 13, wherein the second hook and loop fastener includes two halves, the two halves being disposed longitudinally apart on the second elongate strap such that when the outer side of the other of the end portions is folded over, the two halves are fixed to each other.

15. A method, comprising making the device of claim 1.

16. A method, comprising using the device of claim 1.

17. A method, comprising:
(a) providing a pelvic trauma device, the pelvic trauma device including
a first belt and a second belt;
the first belt including a first elongate strap having a central longitudinal axis and opposing end portions, and a padded enlarged portion having a greater width transverse to the central longitudinal axis than the first elongate strap;
a first fastener that is disposed at at least one of the opposing end portions of the first belt for maintaining a first tension in the first belt;
a second belt including a second elongate strap having a central longitudinal axis, opposing end portions, and a padded enlarged portion having a greater width transverse to the central longitudinal axis than the second elongate strap, the central longitudinal axes of the first and second elongate straps being parallel; and
a second fastener that is disposed at at least one of the opposing end portions of the second belt for maintaining a second tension in the second belt, the second tension being independent of the first tension;
wherein the first and second belts are joined along only a portion of their respective enlarged portions;
(b) placing the lower belt around a greater trochanteric region of a pelvis of a person and tightening the lower belt; and
(c) placing the upper belt around the person at a level of a sacrum and anterior iliac crests and tightening the upper belt independent of tightening the lower belt.

18. The method of claim 17, wherein the step of providing includes providing a posterior pad disposed on only a part of the padded enlarged portion of the first belt, the posterior pad including padding in addition to padding of the padded enlarged portion of the first belt, the method further comprising positioning the posterior pad over the person's posterior sacrum.

19. The method of claim 18, wherein the step of providing includes providing an anterior pad on the upper belt, the method further comprising positioning the anterior pad about 180 degrees from the posterior pad in a midline of an abdomen of the person.

20. A pelvic trauma device, comprising:
a first belt and a second belt;
the first belt including a first elongate strap having a central longitudinal axis and opposing end portions, and a padded enlarged portion having a greater width transverse to the central longitudinal axis than the first elongate strap;
a first fastener that is disposed at at least one of the opposing end portions of the first belt for maintaining a first tension in the first belt;
a posterior pad disposed on a part of the padded enlarged portion of the first belt, the posterior pad including padding in addition to padding of the padded enlarged portion of the first belt, the posterior pad being located on the padded enlarged portion of the first belt so as to contact a wearer's posterior sacrum;
a second belt including a second elongate strap having a central longitudinal axis, opposing end portions, and a padded enlarged portion having a greater width transverse to the central longitudinal axis than the second elongate strap , the central longitudinal axes of the first and second elongate straps being parallel; and
a second fastener that is disposed at at least one of the opposing end portions of the second belt for maintaining a second tension in the second belt, the second tension being independent of the first tension;
wherein the first and second belts are joined only along a portion of their respective padded enlarged portions, the joined portion being no more than one-half an overall length of a longer one of the padded enlarged portions of the first and second belts.
